(19) United States
(12) Patent Application Publication
(10) Pub. No.: US 2009/0035294 A1
(43) Pub. Date: Feb. 5, 2009

(54) LIPOPOLYSACCHARIDE FRACTIONS OF VITREOSCILLA FILIFORMIS USEFUL FOR STIMULATING THE SYNTHESIS OF ANTI-MICROBIAL PEPTIDES OF THE SKIN

(75) Inventors: YANN MAHE, Sainte Genevieve Des Bois (FR); Richard Martin, Rochecorbon (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/055,975

(22) Filed: Mar. 26, 2008

Related U.S. Application Data
(60) Provisional application No. 60/907,507, filed on Apr. 5, 2007.

(30) Foreign Application Priority Data
Mar. 26, 2007 (FR) 0754039

(51) Int. Cl. A61K 31/7028; A61K 38/48; A61K 35/64; A61K 36/02; A61K 36/185; A61P 17/00; A61P 31/00; A61P 31/04; A61P 31/12

(52) U.S. Cl. 424/94.63; 514/42; 424/537; 424/195.17; 424/769

(57) ABSTRACT

Specific fractions of Vitreoscilla filiformis comprising its Lipid A are useful for stimulating the synthesis of anti-microbial peptides of the skin.

### CROSS-REFERENCE TO PRIORITY/PROVISIONAL APPLICATIONS

This application claims priority under 35 U.S.C. § 119 of FR 0754039, filed Mar. 26, 2007, and of U.S. Provisional Application No. 60/907,507, Apr. 5, 2007, each hereby expressly incorporated by reference and each assigned to the assignee hereof.

### BACKGROUND OF THE INVENTION

1. Technical Field of the Invention

The present invention relates to the use of specific fractions of Vitreoscilla filiformis comprising its Lipid A, for stimulating the synthesis of anti-microbial peptides of the skin.

2. Description of Background and/or Related and/or Prior Art

Human skin is made up of two compartments, namely, a superficial compartment, the epidermis, and a deep compartment, the dermis.

The epidermis is composed mainly of three types of cells, which are the keratinocytes (predominant), the melanocytes and the Langerhans cells which play an essential role in the immune response, and in particular in antigen presentation.

The dermis provides the epidermis with a solid support. It is also its feeder element. It is mainly made up of fibroblasts and of an extracellular matrix. Leucocytes, mast cells and tissue macrophages are also found therein. Finally, blood vessels and nerve fibers traverse the dermis.

The skin constitutes a barrier against outside attacks, in particular chemical or mechanical attacks, and, in this respect, a certain number of defense reactions against environmental factors (climate, ultraviolet rays, tobacco, pollution, etc.) and/or xenobiotics (such as, for example, certain medicaments) occur therein. Furthermore, the skin is the seat of allergic reactions, as observed in the case of contact eczema or atopic eczema.

It is known that epidermal cells are capable of responding to microbial (bacterial or fungal) outside attacks by producing peptides and proteins with anti-microbial activity.

These defense peptides and proteases (including beta-defensins, RNAse 7, psoriasin, see the article “Antimicrobial peptides in human skin”, Schröder et al., Mechanisms of epithelial defense, Chem Immunol Allergy, 2005, vol. 86 pp 22-41) are normally produced by the epidermal cells after contact with live intact pathogenic agents.

Under normal conditions, this peptide production is sufficient to limit the proliferation of microorganisms on the skin, thus preventing it from being damaged or infected.

However, situations occur where these systems are not sufficient to limit bacterial proliferation and infections or sepsis of the skin and/or the epithelial mucous membranes are then observed.

The interest in the ability to stimulate this first natural defense system in order to re-establish a natural homeostasis, in particular in situations where the barrier function is deficient, whether partially (dry skin, atopy, etc.) or completely (burns, wounds, dermabrasion, peel treatment), is therefore understandable.

It is thus sought to mimic the activity of whole bacteria on the skin with a compound applied directly to the skin; the use of a mimetic compound in place of a live microorganism makes it possible to avoid any risk of unwanted bacterial proliferation.

It is known, moreover, that Escherichia coli outer membrane lipopolysaccharides (LPS) are capable of mimicking certain of the effects of the whole bacterium so as to stimulate the response of the immune cells via, in particular, the production of “pro-inflammatory” cytokines (such as interleucin 1, TNF-alpha) or else C—C and CXC chemokines such as IL-8 and MCP-1, in order to recruit and activate certain lymphocyte cell populations.

Paradoxically, these E. coli LPSs while they are active systemically or intravenously on the immune cells, are very weakly active, or even inactive, in terms of directly activating the cutaneous first barrier of defense (i.e., the keratinocytes). Thus, they appear to be ineffective for inducing superficial skin defense systems against microorganisms.

### SUMMARY OF THE INVENTION

It has now been demonstrated that a specific fraction of LPS comprising Lipid A of the non-photosynthetic, non-fruiting filamentous bacterium Vitreoscilla filiformis has the ability to stimulate the expression of anti-microbial peptides of the skin by the keratinocytes. Unlike E. coli, this bacterium does not have any pathogenic activity.

This specific LPS fraction has subsequently been evaluated in vitro in comparison with an equivalent fraction of an LPS of E. coli (C14-C16) prepared under the same conditions and at the same final concentration (10 μg/ml), with respect to its possible property of specifically inducing the synthesis of precursors (mRNAs) of epidermal anti-bacterial peptides.

It has thus been observed, surprisingly and unexpectedly, that, compared with E. coli LPS, the specific fraction of Vitreoscilla filiformis Lipid A, at the same concentrations and under the same experimental conditions, is capable of more effectively stimulating the preventive initiating of the skin defense systems, in particular anti-bacterial defense systems.

Thus, this fraction has the biological property of behaving like a “decoy” which activates the skin defense systems without producing the drawbacks thereof related to the intrinsic activity of a pathogenic agent or of a complete bacterium.

Various extracts of Vitreoscilla filiformis having non-specific immunostimulant properties have been described by the assignee hereof with a view to cosmetic or pharmaceutical application, in particular in the treatment of cutaneous signs of aging. This is the case in EP-0,604,631 and EP-0,765,667, which describe, respectively, extracts of non-fruiting filamentous bacteria and a ribosome-rich fraction of these bacteria obtained by centrifugation of the biomass and dialysis of the supernatant obtained.

EP-0,876,813 describes an immunostimulant fraction obtained from the culture medium of these bacteria. WO 94/02158 describes the use of bacterial envelopes or of fractions, in particular LPS fractions, obtained from said envelopes as agents for stimulating the immune system.

Finally, EP-1,400,237 describes an activator that may be a total extract of Vitreoscilla filiformis, for improving the skin's resistance to the non-specific effects of complement activation.

The present invention features the administration of the Lipid A of the non-photosynthetic, non-fruiting filamentous bacterium Vitreoscilla filiformis, as an agent for inducing the expression of anti-microbial peptides by the skin. Lipid A is a part of the LPS fraction.

### DETAILED DESCRIPTION OF BEST MODE AND SPECIFIC/PREFERRED EMBODIMENTS OF THE INVENTION

The Vitreoscilla filiformis bacterial strain (strain deposited with the ATCC 15551) is currently employed by the assignee hereof for the production of a biomass introduced into cosmetic products. The method for preparing this biomass comprises continuous culturing of the bacteria in oxygenated sterile medium, in the presence of mineral salts and of sugars; and sampling followed by centrifugation of the culture medium comprising the bacteria, to obtain a biomass which is placed in flasks and then sterilized. The advantage of processing continuously, and therefore at a constant growth rate μ, is that a physiological state that is itself also constant can be guaranteed. The rupturing of the cells resulting from the sterilization causes decanting of the biomass to a sludge containing essentially cell membranes and coagulated proteins and containing approximately 70% of the weight of LPS, and to a supernatant comprising the cytoplasm and containing approximately 30% of the weight of LPS, the whole bacterium containing (on a dry weight basis) approximately 10% of LPS. A homogeneous biomass is reconstituted by agitation before use.

Usually, the method for preparing the LPS fraction can be carried out according to published conventional techniques.

By way of example, reference is made to the technique known as technique of Westphal et al., (phenol/chloroform/petroleum ether) from 1952 or else the technique of Galanos et al., 1969 (phenol:water), or, alternatively, the technique derived from Westphal: Westphal & Jann [Methods Carbohydr. Chem., 5 (1965) 83], the technique of Galanaos et al. [Eur. J. Biochem., 9 (1969) 245], that of Ni Eidhin and Mouton [FEMS Microbiol. Lett., 110 (1993) 133] and that of Nichols [Infect. Immun., 62 (1994) 3753].

All of these techniques are applicable for extracting an LPS. Nevertheless, none of these techniques leads to a purified fraction.

To prepare a specific fraction including Lipid A, the method of M. Caroff according to WO 2004/062690 entitled “Novel method for isolating endotoxins” can also be used. This method employs a mixture of isobutyric acid and of ammonia in a molar aqueous solution in a ratio by volume of 5/3, or a mixture of isobutyric acid and of triethylamine in an aqueous solution at 10%, in a ratio by volume of 5/3, and the mixture is subsequently stirred for 10 minutes at ambient temperature and then filtered and/or centrifuged at 3000 g for 15 minutes at 4° C. The residual solvent is subsequently eliminated by evaporation under vacuum.

The Lipid A has been characterized after having been isolated from a strain of Vitreoscilla filiformis (EP-1,531,158).

Lipid A of Gram—bacteria is a dimer of glucosamine carrying, through condensation with its hydroxyl groups situated at the 3- and 3′-positions and with its amino groups situated at the 2- and 2′-positions, more or less unsaturated and hydroxylated fatty acids which may themselves be esterified, on their hydroxyl groups, with other fatty acids. It is these fatty acids which allow anchorage of lipid A in the outer membrane of the cell, which is of a phosphoLipid nature. In addition, their nature (as C₁₂-C₁₈) and their position on glucosamines determine the biological activity and the toxicity of the lipids A and therefore of the LPS(s).

According to the invention, the Lipid A has the following structure: (a) the compounds of formula (I):

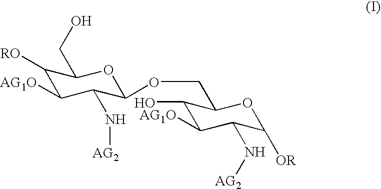

in which:

AG₁ is a 3-hydroxydecanoyl group,

AG₂ is a 3-dodecanoyloxydecanoyl group,

wherein R is a hydrogen atom or a group PO(OR′)₂, in which R′ is a hydrogen atom, a linear or branched, saturated or unsaturated C₁-C₆ alkyl radical, or a phenyl or benzyl group, and

(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that, when more than one groups R, respectively R′, are present, they are identical to or different from one another.

The amine salt of the compound of formula (I) may be selected from the mono-, di- and triethanolamine salts, the mono-, di- or triisopropanolamine salts, 2-amino-2-methyl-1-propanol, 2-amino-2-methyl-1,3-propanediol and tris(hydroxymethyl)aminomethane.

The inorganic salts of the compound of formula (I) may in particular be sodium, magnesium, potassium, zinc or calcium salts.

The Lipid A according to the present invention is either a compound as previously described and that may be obtained according to Example 1, a Lipid A extracted from Vitreoscilla filiformis or a Vitreoscilla filiformis fraction enriched in Lipid A, such a fraction may be a LPS fraction.

The amount of Lipid A in the specific LPS fraction according to the invention will of course depend on the method of extraction employed, the specific LPS fractions rich in Lipid A according hereto comprising at least 0.01% by weight of Lipid A.

The term “skin” means the entire surface of the body, including the skin, the mucous membranes and semi-mucous membranes, the scalp, and also their appendages (nails, body hair, head hair, etc.).

The term “antimicrobial peptides” means peptides expressed in particular by the keratinocytes when they come into contact with microbial pathogenic agents; certain of these peptides have been described by Schröder et al. in anti-microbial peptides in human skin, and comprise beta-defensins, among which are human β-defensin-1, human β-defensin-2, human β-defensin-3, human β-defensin-4, cathelicidin LL-37, serine protease inhibitor anti-leucoprotease, elafin, dermcidin, adrenomedullin, neutrophil gelatinase-associated lipocalin and RNase 7. S100 A7 (or psoriasin) is also one of these peptides with anti-microbial activity that are capable of interfering with the proliferation of bacterial pathogenic agents.

Defensins are anti-microbial peptides that are active on bacteria, fungi and viruses (Harder J. et al., Nature 387: 861, 1997; Frohm M et al., J Biol Chem., 272: 15258-15263, 1997; Gropp R et al., Hum Gene Ther., 10: 957-964, 1999). These peptides are produced by the skin keratinocytes and certain mucosal cells; they can destroy the membrane of the target microbes and/or penetrate their membrane, thereby interfering with the intracellular functions of pathogenic microorganisms. β-defensin 2 and cathelicidin (LL-37) are defensins that can be induced in the skin and an increase therein is brought about during inflammation or stress of the skin (Harder J et al., Nature 387: 861, 1997; Frohm M et al., J Biol Chem., 272: 15258-15263, 1997).

More than about twelve defensins identified at the level of the genes DEFB1; DEFB2; DEFB124; DEFB107, DEF alpha6; DEFB106; DEFB122; DEFB105, DEFB103; DEFB108, DEFalpha4; DEFB125; DEFB124; DEFB104 (also sometimes called DEFB4) are currently known.

The present invention features the utilization of Lipid A of Vitreoscilla filiformis, having the following structure: (a) the compounds of formula (I):

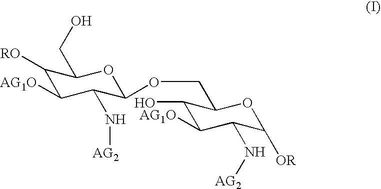

in which:

AG₁ is a 3-hydroxydecanoyl group,

AG₂ is a 3-dodecanoyloxydecanoyl group,

wherein R is a hydrogen atom or a group PO(OR′)₂, in which R′ is a hydrogen atom, a linear or branched, saturated or unsaturated C₁-C₆ alkyl radical, or a phenyl or benzyl group, and

(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that, when more than one groups R, respectively R′, are present, they are identical to or different from one another,

for formulating a composition for use in inhibiting the proliferation of an unwanted microbial, in particular bacterial, flora of the skin, and/or in preventing and/or treating microbial infections and superinfections of the skin.

These infections or superinfections are skin disorders that are in particular linked to the development of pathogenic skin microorganisms selected from bacteria, yeasts or lower mushrooms (fungi) such as, for example, the following bacteria: Staphylococcus aureus, Streptococcus pyogenes, Pseudomonas aeruginosa, Escherichia coli, Clostridium pefringens, Clostridium difficile, Gardnerella vaginalis, Propionibacterium acnes, Klebsiella species, Streptopyogenes, or pathogenic yeasts such as Candida albicans, Malassezia furfur, Trichophyton mentagrophytes, Trichophyton interdigitale, Trichophyton rubrum, Trichophyton yaoundei, Tinea capitis, Tinea corporis, or fungi such as Aspergillus sp.

The skin disorders may be infectious complications of dermatological disorders, in particular acne; infectious complications during cicatrization; dermatophytoses; candidiases; vaginoses; onychomycoses; scalp ringworm, body ringworm; skin disorders linked to therapies with antibiotics or anti-mycotic agents or brought about by hormone disturbances; dermatitis; erysipelas; seborrheic dermatitis.

In particular, the superinfections may be:

superinfected atopic dermatitis, impetiginous eczema, superinfected inflammatory acne, superinfected herpes;

superinfections of skin wounds and lesions irrespective of their origin, in particular during cicatrization and selected from ulcers, wounds and burns;

dermatophytoses such as scalp ringworm, body ringworm, athlete's foot, Hebra's eczema marginatum, herpes circinatus;

candidiases such as mucosal candidiases, vaginal candidiases, interdigital candidiases, candidiases linked to professions at risk or to diabetes;

dermatitis such as impetigo and superficial folliculitis.

This administration may also be to maintain and/or restore a normal ecoflora. The saprophyte microbial flora or ecoflora of the skin is a major factor of immune protection of the skin.

Any imbalance in the population of this flora leads to a functional immunodeficiency and, quite often, occupation of the skin territory by a pathogenic flora.

The skin flora is estimated at 10⁶ cells/cm² (Leyden J J et al., Soc. Inves. Dermatol., 88:65-69, 1987), and consists mainly of corynebacteria (Corynebacterium, Brevibacterium and Propionibacterium) and staphylococci.

Acinetobacter and Micrococcus are also found, but to a lesser degree, with the fungal flora comprising mainly the genus Malassezia (Noble W C, Microbiol Human Skin, London, ed Saunders Company Ltd, 1974; Leeming J P et al, J. Clin. Microbiol., 25: 2017-2019, 1987; Leeming J P et al., J. Applied Bacteriol., 67:47-52, 1989; Rudolf R et al., J. Am. Acad. Dermatol., 1989).

When this ecological barrier of microorganisms is weakened (as in atopic eczema), reduced (in infants), or even destroyed (as after the misuse of aggressive skin products), any unwanted microorganism is then capable of proliferating on the skin or even of crossing it, thus triggering non-specific defense mechanisms.

The presence of the ecoflora thus makes it possible to provide the skin with an ecological line of defense by opposing the implantation of pathogenic microorganisms through a phenomenon of nutritional competition and through the secretion of substances with enzymatic and bactericidal activities.

According to another embodiment, the present invention features the administration of Lipid A of Vitreoscilla filiformis, whether regime or regimen, as an agent for normalizing the skin flora.

The expression “normalizing the skin flora” means maintaining or restoring a normal skin microorganism population profile on the skin, the scalp and/or the mucous membranes, i.e., a population of microorganisms corresponding to that present on normal skin, a normal scalp or a normal mucous membrane.

This invention thus features formulation of a composition comprising at least the Lipid A of Vitreoscilla filiformis, such composition being useful for maintaining and/or restoring a normal skin ecoflora.

In particular, utilization according to the invention may be:

in preventing and/or limiting dandruff conditions. By way of example, the extracts according to the invention may then be formulated in a composition such as a shampoo, a hair lotion or a hair tonic, or a mask to be left on the scalp;

in the formulation of a composition for use in oral hygiene. By way of example, the extracts according to the invention may then be formulated in a composition such as a toothpaste, a chewing gum, an oral mouthwash solution;

in preventing and/or limiting seborrheic states, in particular, in the case of proliferation of pathogenic skin microorganisms which maintain the phenomenon of greasy skin and/or a greasy scalp. By way of example, the extracts according to the invention may then be formulated in care creams such as astringent creams for greasy skin, cleansing compositions for the body, the face or the hair, such as anti-seborrheic shampoos;

in the formulation of a composition for use in body hygiene, in particular intimate hygiene, or hair hygiene. By way of example, the extract according to the invention may then be formulated in cleansing compositions for intimate hygiene or for the body, the face, the hair or the scalp, such as soaps, shower gels, intimate gels, facial cleansing gels, in care products such as anti-bacterial aftershave gels, facial or body milk;

in preventing and/or limiting the unpleasant odors associated with the proliferation of skin microorganisms in the confined areas of the body, for instance the axillary zone or the feet. For this type of application, the extract according to the invention may then be formulated in deodorant compositions.

The use according to the invention may be carried out such that the Lipid A is applied topically to the skin with or without body hair, the scalp, the mucous membranes and/or the semi-mucous membranes.

The oral compositions according to the invention preferably have an ingestible support.

For ingestion, numerous embodiments of oral compositions and in particular of food supplements are possible. Their formulation is carried out by the usual methods for producing sugar-coated tablets, gel capsules, gels, emulsions, tablets or capsules. In particular, the active agent(s) according to the invention may be incorporated into any other forms of enriched foods or food supplements, for example food bars, or compacted or non-compacted powders. The powders may be diluted in water, in soda or in milk or soybean-derived products, or may be incorporated into food bars.

Suitable in particular as food or pharmaceutical supports are milk, yogurt, cheese, fermented milks, milk-based fermented products, ices, fermented cereal-based products, milk-based powders, child and infant formulas, food products of confectionery type, chocolate, cereals, animal feeds, in particular domestic animal feeds, tablets, gel capsules or blocks, oral supplements in dry form.

This application may be carried out on any mammal, preferably humans and pets or farm animals.

These applications may be extended to any composition for use in veterinary treatments. They may also be more specifically for use in the prevention of microbial, in particular bacterial, proliferations. By way of example which is not limiting in nature, it may involve an application on the udder of cows and, in general, of any milk-producing mammals.

This veterinary application makes it possible to treat and/or prevent disorders linked to staphylococcal, streptococcal and mycotic infections.

For all these uses, it is advantageous to use the Lipid A of Vitreoscilla filiformis in combination with probiotics and/or prebiotics.

The probiotic microorganisms suitable for the invention are microorganisms which can be administered without any risks to animals or humans.

For the purpose of the present invention, the term “probiotic microorganism” (or else probiotic) means a live microorganism which, when it is consumed in adequate amount, has a positive effect on the health of its host “Joint FAO/WHO Expert Consultation on Evaluation of Health and Nutritional Properties of Probiotic in Food Including Powder Milk with Live Lactic Acid Bacteria, 6 Oct. 2001”, and which can, in particular, improve the intestinal microbial balance.

According to one embodiment of the invention, this microorganism is used in an isolated form, i.e., not mixed with one or more compound(s) that may be associated with it in its environment of origin.

For the purpose of the invention, the term “fraction” is more particularly a fragment of said microorganism which is effective in the treatment of sensitive and/or dry skin by analogy with said whole microorganism.

The microorganisms suitable for the invention may be selected, in particular, from ascomycetes such as Saccharomyces, Yarrowia, Kluyveromyces, Torulaspora, Schizosaccharomyces pombe, Debaromyces, Candida, Pichia, Aspergillus and Penicillium, and bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus, and mixtures thereof.

As ascomycetes most particularly suitable for the present invention, particularly exemplary are Yarrowia lipolitica and Kluyveromyces lactis, and similarly Saccharomyces cereviseae, Torulaspora, Schizosaccharamyces pombe, Candida and Pichia.

As regards the probiotic microorganisms, the following bacterial and yeast genera are generally employed:

lactic acid bacteria: which produce lactic acid by fermentation of sugar. They are divided into two groups according to their morphology:

Lactobacillus species: Lactobacillus acidophilus; amylovorus, casei, rhamnosus, brevis, crispatus, delbrueckii (subsp bulgaricus, lactis), fermentum, helveticus, gallinarum, gasseri johnsonii, paracasei, plantarum, reuteri, salivarius, alimentarius, curvatus, casei subsp. casei, sake,

Gocci: Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus camosus, Staphylococcus xylosus;

bifidobacteria or Bifidobacterium species: Bifidobacterium adolescentis, animalis, bifidum, breve, lactis, longum, infantis, pseudocatenulatum;

yeasts: Saccharomyces (cerevisiae or else boulardii);

the other sporulated bacteria: Bacillus (cereus var toyo or subtilis), Bacillus coagulans, Bacillus licheniformis, Escherichia coli strain nissle, Propionibacterium freudenreichii;

and mixtures thereof.

Lactic acid bacteria and bifidobacteria are the probiotics most commonly used.

Specific examples of probiotic microorganisms are Bifidobacterium adolescentis, Bifidobacterium animalis, Bifidobacterium bifidum, Bifidobacterium breve, Bifidobacterium lactis, Bifidobacterium longum, Bifidobacterium infantis, Bifidobacterium pseudocatenulatum, Lactobacillus acidophilus (NCFB 1748); Lactobacillus amylovorus, Lactobacillus casei (Shirota), Lactobacillus rhamnosus (strain GG), Lactobacillus brevis, Lactobacillus crispatus, Lactobacillus delbrueckii (subsp bulgaricus, lactis), Lactobacillus fermentum, Lactobacillus helveticus, Lactobacillus gallinarum, Lactobacillus gasseri, Lactobacillus johnsonii (CNCM I-1225), Lactobacillus paracasei, Lactobacillus plantarum, Lactobacillus reuteri, Lactobacillus salivarius, Lactobacillus alimentarius, Lactobacillus curvatus, Lactobacillus casei subsp. casei, Lactobacillus sake Lactococcus lactis, Enterococcus (faecalis, faecium), Lactococcus lactis (subspp lactis or cremoris), Leuconstoc mesenteroides subsp dextranicum, Pediococcus acidilactici, Sporolactobacillus inulinus, Streptococcus salvarius subsp. Thermophilus, Streptococcus thermophilus, Staphylococcus carnosus, Staphylococcus xylosus, Saccharomyces (cerevisiae or else boulardii), Bacillus (cereus var toyo or subtilis), Bacillus coagulans, Bacillus licheniformis, Escherichia coli strain nissle, Propionibacterium freudenreichii, and mixtures thereof.

The microorganisms may be formulated in the powdered state, i.e., in a dry form, or in the form of suspensions or of solutions.

More particularly, the microorganisms are probiotic microorganisms derived from the group of lactic acid bacteria, such as, in particular, Lactobacillus and/or Bifidobacterium. By way of illustration of these lactic acid bacteria, mention may more particularly be made of Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

The species most particularly suitable are Lactobacillus johnsonii, Lactobacillus paracasei, Bifidobacterium adolescentis, Bifidobacterium longum and Bifidobacterium lactis NCC 2818 respectively deposited according to the Treaty of Budapest with the Institut Pasteur (28 rue du Docteur Roux, F-75024 Paris cedex 15) on 30/06/92, 12/01/99, 15/04/99, 15/04/99, 07/06/05 under the following designations: CNCM I-1225, CNCM I-2116, CNCM I-2168, CNCM I-2170 and CNCM I-3446, and the genus Bifidobacterium longum (BB536), and mixtures thereof.

The microorganisms and/or their fractions and/or metabolites can be formulated in a suitable support in an amount equivalent to at least 10³ cfu/g, in particular at doses ranging from 10⁵ to 10¹⁵ cfu/g, and more particularly from 10⁷ to 10¹² cfu/g of support.

The compositions for topical application or for oral administration according to the invention generally comprise from 10³ to 10¹² cfu, in particular from 10⁵ to 10¹⁰ cfu, and more particularly from 10⁷ to 10⁹ cfu of microorganisms, in particular probiotic microorganisms, per gram of support.

The microorganism(s) may be included in the composition according to the invention in a live, semi-active or inactivated, or dead form.

It (they) may also be included in the form of fractions of cellular components or in the form of metabolites. The microorganism(s), metabolite(s) or fraction(s) may also be introduced in the form of a lyophilized powder, of a culture supernatant and/or, where appropriate, in a concentrated form.

According to a specific embodiment, the microorganisms are used in an inactivated or even dead form, in particular in the topical compositions.

The term “prebiotic” means a non-digestible dietary component capable of selectively stimulating the growth and/or the activity of the probiotic bacteria normally present in the colon, so as to have a beneficial effect on the organism.

In general, oligosaccharides are the main sources of prebiotics.

In this respect, all FOSs (fructooligosaccharides) are prebiotics which stimulate the growth of Bifidobacteria. Inulin and its derivatives, found in chicory (Ciichorium intybus) and Jerusalem artichoke (Helianthus tuberosus) and in Compositae or in Campanulaceae, are also promoters of Bifidobacteria. Isomaltooligosaccharides such as isomaltose, initially, but also panose, isomaltotrealose, kojibiose, etc., are all excellent stimulants of the growth of Bifidobacteria and of lactobacilli.

The administration according to the invention is found to be particularly advantageous when the Lipid A of Vitreoscilla filiformis is administered in combination with peeling agents or after application of peeling agents, for stimulating the endogenous defences of the skin by the keratinocytes.

Thus, this invention also features a cosmetic peeling process comprising application before, simultaneously with or after the application of a peeling and/or desquamating agent, preferably simultaneously with or after.

Peels are a well known means for improving the appearance and/or the texture of the skin and/or of the scalp, in particular improving the radiance and the homogeneity of the complexion and/or reducing the visible and/or tactile irregularities of the skin, and in particular for improving the surface appearance of the skin, for reducing actinic lentigo, acne marks and chickenpox marks, and also for preventing, reducing or combating the signs of skin aging, and in particular for smoothing out the irregularities of the skin's texture, such as wrinkles and fine lines.

They have the effect of removing a superficial part of the skin to be treated (epidermis and possibly superficial layer of the dermis) by chemical methods.

A consequence of the effect of peeling or desquamating agents is also the elimination of a part of the natural ecoflora of the skin. The concomitant use of the LPS fraction according to the invention makes it possible to prevent the development of an unwanted flora in place of the ecoflora of the skin.

The term “peeling agent” or “desquamating agent” means any compound capable of acting:

either directly on desquamation by promoting exfoliation, such as: saturated and unsaturated monocarboxylic acids, saturated and unsaturated dicarboxylic acids, saturated and unsaturated tricarboxylic acids; alpha-hydroxy acids and beta-hydroxy acids of monocarboxylic acids; alpha-hydroxy acids and beta-hydroxy acids of dicarboxylic acids; alpha-hydroxy acids and beta-hydroxy acids of tricarboxylic acids; keto acids, alpha-keto acids, beta-keto acids of polycarboxylic acids, of polyhydroxymonocarboxylic acids, of polyhydroxydicarboxylic acids or of polyhydroxytricarboxylic acids; and (3-hydroxy-2-pentylcyclopentyl)acetic acid.

Preferred α-hydroxy acids that are exemplary include: glycolic acid, citric acid, lactic acid, tartaric acid, malic acid or mandelic acid.

Preferred β-hydroxy acids are selected from: salicyclic acid and derivatives thereof, in particular 5-n-octanoylsalicylic acid.

Other exfoliants that are exemplary include: dioic acid, pyruvic acid, gluconic acid, glucuronic acid, oxalic acid, malonic acid, succinic acid, acetic acid, gentisic acid, cinnamic acid, azelaic acid; phenol, resorcinol; urea and derivatives thereof; oligofucoses as in EP-0,218,200; jasmonic acid and derivatives thereof as in EP-1,333,022 and EP-1,333,021; trichloroacetic acid; retinoids such as retinol or retinoic acid; adapalene; extract of Saphora japonica; resveratrol; and also salts and derivatives thereof, such as the cis or trans forms, racemic mixtures and the dextrorotatory or laevorotatory forms of the abovementioned agents;

or on the enzymes involved in desquamation or degradation of corneodesmosomes, such as glycosidases, stratum corneum chymotryptic enzyme (SCCE) or even other proteases (trypsin, chymotrypsin-like). Particularly exemplary are mineral salt chelating agents such as EDTA; N-acyl-N,N′,N′-ethylenediaminetriacetic acid; aminosulfonic compounds, and in particular (N-2-hydroxyethylpiperazine-N-2-ethane)sulfonic acid (HEPES); derivatives of 2-oxothiazolidine-4-carboxylic acid (procysteine); derivatives of alpha-amino acids of glycine type (as described in EP-0,852,949, and also sodium methyl glycine diacetate marketed by BASF under the trademark Trilon M®); honey; sugar derivatives such as O-octanoyl-6-D-maltose, O-linoleyl-6-D-glucose and N-acetylglucosamine.

As other desquamating agents that can be included in the compositions according to the invention, exemplary are extracts of laminaria such as Laminaria saccharina and Laminaria ochrolenca, glyceryl trilactate, siliceous salicylate derivatives as in EP-0,796,861, 5-acylsalicylic acid salts, active agents with effects on transglutaminase as in EP-0,899,330, and an extract of Ficus opuntia indica blossom, such as Exfolactive® from Silab.

Those skilled in the art will be able to define the required amount of desquamating agent present in the combinations according to the invention in order to obtain the desired effect on the skin.

By way of example, the desquamating agent may be in an amount ranging from 0.001% to 95% by weight relative to the total weight of the composition, in particular from 0.01% to 30% by weight relative to the total weight of the composition, preferably from 0.01% to 10% by weight relative to the total weight of the composition.

The present invention also features a cosmetic skin-peeling process comprising the application, simultaneously with or following the application of peeling agent, of the Lipid A of Vitreoscilla filiformis. Advantageously, said Lipid A is formulated in a cosmetic composition.

This Lipid A of Vitreoscilla filiformis comprising Lipid A also finds advantageous applications in skin model engineering.

Thus, the invention also features the use of the Lipid A of Vitreoscilla filiformis, for preventing microbial proliferation in cell or organotypic cultures. This use may be as a replacement for or in combination with natural or synthetic antibiotics.

Such a use allows the septic preparation of epidermal and/or skin models or else the septic preparation of skin explants or of hair grafts.

The skin models may be epidermises generated from epithelial cells, keratinocytes, progenitors, stem cells.

They may also and non-exclusively be epidermises reconstructed on a synthetic membrane, on a collagen biosupport, or on a native acellular de-epidermalized dermis (DED model), or live dermises comprising fibroblasts included in a collagen gel (lattice model) or included in a collagen sponge.

The reconstructed skin models may also comprise non-keratinocyte cells such as Langerhans cells and/or melanocytes, integrated into the epidermis, and/or non-fibroblast cells such as endothelial cells and/or lymphocytes, integrated into the dermis.

The skin models can also be explants maintained under survival conditions ex vivo.

By way of example, representative is the reconstructed epidermis model EpiSkin®, obtained by culturing adult human keratinocytes on a collagen support under conditions which allow their terminal differentiation and the reconstruction of an epidermis with a functional horny layer.

The present invention may be applied to the various skin models commercially available: EpiSkin®, and also in particular, and non-exclusively, SkinEthic®, Matek® or Natskin®.

Various models of reconstructed epidermis and/or reconstructed skin (experimental research models and/or commercially available models) are in particular described in the following references: Asselineau D, Bernard B, Bailly C, Darmon M (1985). Epidermal morphogenesis and induction of the 67 kD keratin polypeptide by culture of human keratinocytes at the liquid-air interface. Exp. Cell. Res., 159: 536-539.; Bernerd F, Vioux C, Lejeune F, Asselineau D (2003). The sun protection factor (SPF) inadequately defines broad spectrum photoprotection: demonstration using skin reconstructed in vitro exposed to UV-A, UV-B or UV-solar simulated radiation. Eur. J. Dermatol., 13: 242-249; Lenoir M C, Bernard B A, Pautrat G, Darmon M, Shroot B (1988). Outer root sheath cells of human hair follicle are able to regenerate a fully differentiated epidermis in vitro. Dev. Biol., 130: 610-620; Prunieras M, Regnier M, Wooodley D (1983). Methods for cultivation of keratinocytes with an air-liquid interface. J. Invest. Dermatol., 81 (1 suppl.): 28s-33s; Regnier M, Staquet M J, Schmitt D, Schmidt R (1997). Integration of Langerhans cells into a pigmented reconstructed human epidermis. J. Invest. Dermatol., 109: 510-512; Regnier M, Desbas C, Bailly C, Darmon M (1988). Differentiation of normal and tumoral human keratinocytes cultured on dermis: reconstruction of either normal or tumoral architecture. In Vitro Cell. Dev. Biol., 24: 625-632; Regnier M, Schweizer J, Michel S, Bailly C, Prunieras M (1986). Expression of high molecular weight (67K) keratin in human keratinocytes cultured on dead de-epidermized dermis. Exp. Cell. Res., 165: 63-72; Tinois E, Tiollier J, Gaucherand M, Dumas H, Tardy M, Thivolet J (1991). In vitro and post-transplantation differentiation of human keratinocytes grown on the human type IV collagen film of a bilayered dermal substitute. Exp. Cell. Res., 193: 310-319.

The process according to the invention may also be carried out on other models of epithelium. These may in particular, and non-exclusively, be models of oral, gingival, oesophageal, vaginal, corneal (ocular) or alveolar (pulmonary) epithelium, such as the SkinEthic® commercial models.

Various models of epithelium are in particular described in the following references:

Oral and gingival: Jayatilake JAMS, Samaranayake Y H, Samaranayake L P (2005). An ultrastructural and a cytochemical study of candidal invasion of reconstituted human oral epithelium. J. Oral. Pathol. Med., 34: 240-246; Vande Vannet B, Hanssens J-L (2004). Characterization of human oral and gingival mucosal models: a histological characterization and applications in toxicity testing. 3^{rd} International SkinEthic Workshop, Nice, France.

Oesophageal: Bernhardt J, Bernhardt H, Knoke M, Ludwig K (2004). Influence of voriconazole and fluconazole on reconstituted multilayered oesophageal epithelium infected by Candida albicans. Mycoses, 47, 7, p 330.

Vaginal: Schaller M, Korting H C, Borelli C, Hamm G, Hube B (2005). Candida albicans-secreted aspartic proteinases (Sap) modify the epithelial cytokine response in an in vitro model of vaginal candidiasis. Infect. Immun., 73: 2758-2765.

Corneal: Van Goethem F, Adriaens E, Alépée N, Straube F, De Wever B, Cappadoro M, Catoire S, Hansen E, Wolf A, Vanparys P (2006). Prevalidation of a new in vitro reconstituted human corneal model to assess the eye irritating potential of chemicals. Toxicology In Vitro., 20:1-17.

Alveolar: Roggen E, Nilsson N, Dam L, Mikkelsen B, Rasmussen C, Cappadoro M, Rosdy M (2001). The use of airlifted reconstituted human lung epithelial tissues to predict in vitro the allergenic risk of topically applied enzyme proteins. Congrès de la Société de pharmaco-toxicologie cellulaire (SPTC), Toulouse, France.

Preferably, the Lipid A of Vitreoscilla filiformis will be formulated in a cosmetic or pharmaceutical composition suitable to be applied topically to the skin, the scalp or the mucous membranes.

The compositions comprise from 0.001 ng/ml to 100 mg/ml, and preferably from 1 ng/ml to 100 μg/ml of Lipid A of Vitreoscilla filiformis.

The compositions according to the invention may be in any of the forms suitable for the applications envisaged, in particular topical applications, in the cosmetics and dermatological fields.

The compositions according to the invention may thus be applied topically to any cutaneous area of the body, the skin, the scalp or the mucous membranes and may be in any galenical form adapted by those skilled in the art. They may in particular be in the form of an aqueous or oily solution or suspension, an oil-in-water or water-in-oil or multiple emulsion, a silicone-based emulsion, a microemulsion or a nanoemulsion, an aqueous or oily gel or an anhydrous liquid, pasty or solid product.

For topical application to the skin, the composition may have the form in particular of an aqueous or oily solution or a dispersion of the lotion or serum type, emulsions with a liquid or semi-liquid consistency of the milk type, obtained by dispersion of a fatty phase in an aqueous phase (O/W) or conversely (W/O), or suspensions or emulsions with a soft consistency of the aqueous or anhydrous gel or cream type, or else microcapsules or microparticles, or vesicular dispersions of the ionic and/or non-ionic type. It may also be in the form of a patch or of a controlled-release system. These compositions are prepared according to the usual methods.

They may also be used for the hair in the form of aqueous, alcoholic or aqueous-alcoholic solutions, in the form of creams, gels, emulsions or mousses, or else in the form of aerosol compositions also comprising a pressurized propellant.

The amounts of the various constituents of the compositions according to the invention are those conventionally used in the fields under consideration.

These compositions constitute, in particular, cleansing, protective, treating or care creams for the face, for the hands, for the feet, for the major anatomical folds or for the body (for example day creams, night creams, makeup-removing creams, foundation creams, anti-sun creams), masks to be left in on the skin or the hair, fluid foundations, makeup-removing milks, protective or care body milks, anti-sun milks, skincare lotions, gels or mousses, for instance cleansing lotions, anti-sun lotions, artificial tanning lotions, bath compositions, deodorizing compositions comprising a bactericidal agent, aftershave gels or lotions, hair-removing creams, compositions for treating certain skin diseases such as acne, eczema or psoriasis.

The compositions according to the invention may also be solid preparations constituting soaps or cleansing bars.

The compositions may also be packaged in the form of an aerosol composition also comprising a pressurized propellant.

The compositions according to the invention may also be hair care compositions, and in particular a shampoo, a hair setting lotion, a medicated lotion, a styling cream or gel, a dye composition (in particular for oxidation dyeing), optionally in the form of coloring shampoos, hair restructuring lotions, a permanent wave composition (in particular a composition for the first stage of a permanent wave operation), a lotion or gel for combating hair loss, an anti-parasitic shampoo, etc.

The compositions may also be for orodental use, for example a toothpaste. In this case, the composition may contain adjuvants and additives that are normal for compositions for oral use, and in particular surfactants, thickeners, humectants, polishing agents such as silica, various active ingredients such as fluorides, in particular sodium fluoride, and optionally sweeteners such as sodium saccharinate.

When the composition is an emulsion, the proportion of the fatty phase may range from 5% to 80% by weight, and preferably from 5% to 50% by weight, relative to the total weight of the composition. The oils, the waxes, the emulsifiers and the coemulsifiers used in the composition in emulsion form are selected from those conventionally used in the cosmetics field. The emulsifier and the coemulsifier are present, in the composition, in a proportion ranging from 0.3% to 30% by weight, and preferably from 0.5% to 20% by weight, relative to the total weight of the composition. The emulsion may also contain Lipid vesicles.

When the composition is an oily solution or gel, the fatty phase may represent more than 9% of the total weight of the composition.

In a known manner, the cosmetic composition may also contain adjuvants that are normal in the cosmetics field, such as hydrophilic or lipophilic gelling agents, hydrophilic or lipophilic additives, preservatives, antioxidants, solvents, fragrances, fillers, screening agents, odor absorbers and dyestuffs. The amounts of these various adjuvants are those conventionally used in the cosmetics field, and for example from 0.01% to 10% of the total weight of the composition. Depending on their nature, these adjuvants may be introduced into the fatty phase, into the aqueous phase and/or into the Lipid spherules.

As oils or waxes that can be used in the invention, exemplary are mineral oils (liquid petroleum jelly), plant oils (liquid fraction of shea butter, sunflower oil), animal oils (perhydrosqualene), synthetic oils (purcellin oil), silicone oils or waxes (cyclomethicone) and fluoro oils (perfluoropolyethers), beeswax, carnauba wax or paraffin wax. Fatty alcohols and fatty acids (stearic acid) may be added to these oils.

As emulsifiers according to the invention, exemplary are glyceryl stearate, polysorbate 60 and the mixture of PEG-6/PEG-32/glycol stearate marketed under the trademark Tefose® 63 by Gattefosse.

Exemplary solvents are lower alcohols, in particular ethanol and isopropanol, and propylene glycol.

Exemplary hydrophilic gelling agents according to the invention include carboxyvinyl polymers (carbomer), acrylic copolymers such as acrylate/alkyl acrylate copolymers, polyacrylamides, polysaccharides such as hydroxypropylcellulose, natural gums and clays, and exemplary lipophilic gelling agents include modified clays such as Bentones®, metal salts of fatty acids such as aluminum stearates, and hydrophobic silica, ethylcellulose and polyethylene.

When Lipid A is contained in a specific LPS fraction (enriched in Lipid A), the amount of LPS fraction of Vitreoscilla filiformis present in the compositions according to the invention will be adjusted by those skilled in the art so as to obtain the regulatory activity. By way of indication, the amount of specific LPS fraction of Vitreoscilla filiformis in the compositions will be from 0.0001% to 10% by weight relative to the total weight of the composition, preferably from 0.001% to 2%; it will in particular be at least 0.01%.

Advantageously, the composition according to the invention will comprise nanospheres or microspheres, as described, for example, in EP-0,447,318, EP-0,557,489, EP-1,151,741, EP-1,201,219 or WO 97/12602.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Preparation of Lipid A

Synthesis of A:

A solution containing commercial glucosamine A′ (1.4 mmol) and 3-dodecanoyloxydecanoyl (1.54 mmol) in CH₂Cl₂ (15 ml) is treated with EDC.MeI (2.10 mmol) and stirred at room temperature overnight. The reaction mixture is then concentrated and the residue is purified by flash chromatography on silica gel.

The above compound (15.2 mmol) is dissolved with 3-hydroxydecanoyl (16.7 mmol) and 4-pyrrolidinopyridine (1.7 mmol) in CH₂Cl₂ (95 ml). EDC.MeI (16.7 mmol) is added and the medium is stirred at room temperature overnight. The reaction mixture is then concentrated and the residue is purified by flash chromatography on silica gel.

The acetonide obtained above is dissolved in an acetic acid/water (4/1) mixture and heated at 60° C. for 1 h. The medium is then concentrated and purified by flash chromatography on silica gel to give the intermediate A.

b) Synthesis of B:

B′ is obtained from the commercial product B″ by applying the same protocol used for the synthesis of A (see above).

TCBOC-CI (13.2 mmol) in CH₂Cl₂ (25 ml) is added dropwise over 15 min to a solution at 0° C. containing B′ (12 mmol) and pyridine (25 mmol) in CH₂Cl₂ (125 ml). The mixture is then gently adjusted to room temperature over 3.5 h. 4-Pyrrolidinopyridine (6.0 mmol), N,N-diisopropylethylamine (60 mmol) and diphenyl chlorophosphonate (18 mmol) are added successively. The mixture is stirred at room temperature for 5 h. The reaction is then diluted with CH₂Cl₂, washed with a cold aqueous HCl solution (7.5%) and then with a saturated aqueous NaHCO₃ solution, and then dried and concentrated. The residue is purified by flash chromatography on silica gel.

ZnCl₂ (1.0 M in ether, 2.41 mmol) is added at 0° C. to a solution containing the compound obtained above (4.84 mmol) and dichloromethyl methyl ether (24.2 mmol) in CHCl₃ (60 ml). The mixture is gently adjusted to room temperature, and then stirred at room temperature overnight. The reaction medium is then diluted with EtOAc, washed with a saturated aqueous NaHCO₃ solution and then dried and concentrated. The residue is purified by flash chromatography on silica gel to give compound B.

c) Synthesis of C:

A solution containing B (1.85 mmol) and A (1.54 mmol) in 1,2-dichloroethane (18.5 mmol) is stirred with 4 Å molecular sieve (1 g) for 1 h, and then treated with AgOTf (5.55 mmol) in a single portion. After having stirred for 4 h at room temperature in the dark, additional AgOTf (1.85 mmol) is added and the reaction is stirred overnight. The creamy mixture is then filtered on Celite and concentrated. Purification by flash chromatography on silica gel makes it possible to obtain compound C′.

A solution containing C′ (0.46 mmol) in an AcOH (4.5 ml)/THF (40 ml) mixture is hydrogenated in the presence of PtO₂ (0.45 g) at room temperature at a pressure of 70 psig for 18 h. The solution is diluted with CHCl₃/MeOH (2/1), and then briefly sonicated. The catalyst is then filtered and washed with the CHCl₃/MeOH (2/1) mixture. The filtrates are grouped together and concentrated. The residue is purified by flash chromatography on silica gel.

The preceding product (12 mmol) in CH₂Cl₂ (25 ml) is stirred in the presence of N,N-diisopropylethylamine (60 mmol) and POCl₃ (18 mmol) at room temperature for 5 h. The reaction is then concentrated. The reaction medium is then diluted with CH₂Cl₂, washed with a saturated aqueous NaHCO₃ solution, and then dried and concentrated. The residue is then dissolved in acetic acid (100 ml) and heated to 60° C. with zinc dust (0.9 ml). The reaction is then cooled and filtered on Celite, and then concentrated. Purification by flash chromatography on silica gel makes it possible to obtain compound C.

### Example 2

### Preparation of an Extract of Vitreoscilla filiformis Containing Lipid A

125 grams of Vitreoscilla filiformis lyophilizate are obtained as follows:

continuous-mode culturing (μ=0.12H−1) in an efficient 3 m³ fermenter equipped with a draft tube; continuous harvesting by centrifugation (10 000 G) under sterile conditions;

lyophilization of the biomass thus obtained.

These 125 grams of lyophilized biomass are subsequently extracted with the following mixture:

33.5 ml of concentrated NH₄OH;

906 ml of osmosed water;

1560 ml of isobutyric acid.

The extraction is carried out for 10 to 30 minutes with stirring at ambient temperature.

The product obtained is centrifuged at 8000 G/30 min/4° C. in order to remove the particles. The centrifugation supernatant is subsequently filtered through a GFD then GFF filter.

NB: The pellet is the Vitreoscilla filiformis biomass with 95% of the LPS removed (fraction subsequently referred to as LPS free, used in Example 4).

The whole is then evaporated under vacuum at 65° C./40 mbar.

A 600 ml concentrate is obtained and is placed in a ventilated oven in order to be concentrated. 37 g of a paste are then obtained, which paste, with 50% of water added, is lyophilized.

### Example 3

### Measurement of the Induction of the Expression of Skin Defense Enzymes by Vitreoscilla filiformis Lipid A, Comparison with E. coli LPS

Two extracts of purified bacterial lipopolysaccharides, one corresponding to the extract according to the invention (specific LPS fraction rich in Lipid A), the other being an LPS extract of Escherichia coli of commercial origin, were evaluated, by means of a genomic analysis (Affymetrix U133 plus arrays), for their ability to specifically induce the expression of particular genes, in the skin cells, encoding these anti-bacterial proteins.

Normal human epidermal keratinocytes (NHEKs) are precultured in SFM culture medium (Invitrogen) with epidermal growth factor at 0.25 ng/ml and pituitary extracts at 25 μg/ml (EGF, EP, Invitrogen 3700015) and gentamycin at 25 μg/ml (Sigma G1397), and then placed in test medium.

They are subsequently introduced into test medium (same medium as for the culturing, with neither EP nor EGF).

The cells are subsequently treated or not treated (control) with the test products and cultured for 24 hours at 37° C. and 5% CO₂.

The culture supernatants were subsequently removed and the carpets of cells were rinsed with a solution of PBS and then placed in sterile RNA-free tubes in the presence of tri-reagent (Sigma T9424) and immediately frozen at −80° C.

The expression of the precursors of synthesis of the proteins of interest was evaluated by RT-Q-PCR on the messenger RNAs extracted from the carpets of cells from each treatment:

### Reverse Transcription:

extracting the total RNA of each sample using tri-reagent according to the protocol recommended by the supplier;

eliminating the traces of potentially contaminating DNA by treatment with the DNA-free system (Ambion ref. 1906);

carrying out the mRNA reverse-transcription reaction in the presence of the oligo(dT) primer and of the SuperscriptII enzyme (Invitrogen 18064071) for the Q/RT PCR studies (Example 3) or by means of the Affymetrix one cycle reverse transcription kit for the Affymetrix transcriptional studies (Example 2).

By means of a biostatistical analysis coupled to a data mining analysis on the entire human genome, it was thus observed that, contrary to E. coli LPS, which is known to have little stimulatory activity on skin keratinocytes, the LPS comprising Lipid A from Vitreoscilla filiformis, at the same concentrations and under the same experimental conditions, was capable of fully stimulating the preventive setting in motion of the defense systems of the skin, in particular anti-bacterial defense systems.

Thus, this bacterial or mimetic constituent has the biological property of inducing the defense systems of the skin, in particular the production of beta-defensin and cathelicidin, but also of protease inhibitors such as elafin (SKALP); Elias et al., Exp. Dermatol., (14): 719-726.

Thus, as indicated in the table below, in response to V. filiformis Lipid A, the induction of mRNA encoding beta-defensin-2, RNase7 and PS100A7, but also other anti-proteases which contribute to epidermal defense, is clearly observed.

| | | Specific LPS | |
|---|---|---|---|
| | | Fraction of | |
| | | Vitreoscilla | |
| | | filiformis | |
| | | According to the | |
| | | Invention | |
| | | (rich in Lipid A) | LPS Fraction |
| | | on Normal | of E. coli on |
| Genbank | | Human | Normal Human |
| References | Description | Keratinocytes | Keratinocytes |
| NM_002963 | psoriasin 1 (S100 | 14.8 | 1.4 |
| | calcium binding | | |
| | protein A7) | | |
| AJ243672 | S100 calcium binding | 14.7 | 1.2 |
| | protein A7-like 1 | | |
| NM_004942 | defensin beta 2 | 8.6 | 1.1 |
| AJ131212 | ribonuclease, RNase A | 2.6 | −1.0 |
| | family, 7 | | |
| AI554300 | serine (or cysteine) | 3.5 | 1.3 |
| | proteinase inhibitor, | | |
| | clade B (ovalbumin), | | |
| | member 1 | | |
| NM_002638 | protease inhibitor 3, | 3.5 | 1.3 |
| | skin-derived | | |
| | (SKALP)///protease | | |
| | inhibitor 3, skin- | | |
| | derived (SKALP) | | |
| NM_030666 | serine (or cysteine) | 3.4 | 1.2 |
| | proteinase inhibitor, | | |
| | clade B (ovalbumin), | | |
| | member 1 | | |
| L10343 | protease inhibitor 3, | 2.7 | 1.2 |
| | skin-derived (SKALP) | | |

### Example 4

### Comparison of the Expression of Beta-Defensin-2 by Keratinocytes Using the Fraction According to the Invention, a Fraction of Vitreoscilla filiformis without LPS and an LPS Fraction of E. coli

The PCR reactions (polymerase chain reactions) were carried out by quantitative PCR with the “Light Cycler” system (Roche Molecular Systems Inc.) and according to the procedures recommended by the supplier.

The reaction mix (10 μl final volume) for each sample is the following:

2.5 μl of cDNA diluted to 1/10^{th};

primers for the various markers used;

reaction mix (Roche) containing the taq DNA polymerase enzyme, the SYBR Green I marker (fluorophore which intercalates into the double-stranded DNA during the elongation step) and MgCl₂.

Analysis by Q-PCR:

The incorporation of fluorescence into the amplified DNA is measured continuously during the PCR cycles. This system makes it possible to obtain curves of fluorescence measurement as a function of PCR cycles and to thus evaluate a relative expression value for each marker.

The number of cycles is determined from “exit” points on the fluorescence curves. For the same marker analyzed, the later a sample exits (high number of cycles), the lower the initial number of copies of mRNA.

The RE (relative expression) value is expressed in arbitrary units according to the following formula: ½^{number of cycles}×10⁶.

The protocol for preparing the RNAs of Example 3 was carried out with two other products:

a fraction of Vitreoscilla filiformis without LPS (fraction termed LPS free, see Example 2) as noted in the preparation of the V. filiformis LPS. It is the total biomass (proteins, etc.) with 95% of its LPS removed;

a commercial LPS fraction of Escherichia coli.

| | | Expression of |
|---|---|---|
| | | Beta-Defensin-2 |
| Treatment | Concentration | % Control |
| Control | — | 100 |
| Fraction according to the | 10 μg/ml | 221 |
| invention (prepared | | |
| according to Example 1) | | |
| Fraction A | 0.1% | 155 |
| Fraction B | 10 μg/ml | 191 |

### Example 5

### Compositions

Oral Administration:

### Example 1

### Single-Dose Powder Sachet

| Active ingredient | mg/sachet |
|---|---|
| Extract with Lipid A of Vitreoscilla filiformis | 1 |
| (example 2) | |
| Magnesium citrate in mg of magnesium | 300 |
| Inulin | 50 |
| Borage oil | 100 |
| Walnut oil | 100 |
| Excipient | |
| Maltodextrin | Qs |
| Sodium benzoate | Qs |

One to three sachets per day may be taken.

### Example 6

### Capsule

| | mg/capsule |
|---|---|
| Lactobacillus paracasei (CNCM I-2116) | 50.00 |
| Extract with Lipid A of Vitreoscilla filiformis | 5.00 |
| (Example 2) | |
| Blackcurrant seed oil | 100.00 |
| Fish oil | 100.00 |
| Magnesium stearate | 0.02 |
| Natural flavor | Qs |
| Excipient | Qs |

One to three of these capsules can be taken per day.

B. Topical Administration to the Skin:

### Example 7

### Deodorant Spray

### Pump-Dispenser Bottle

| | |
|---|---|
| Extract with Lipid A of Vitreoscilla filiformis (Example 2) | 0.30 g |
| Fragrance, dyes | Qs |
| Ethyl alcohol 95 vol. % denatured | 100 g |

### Example 8

### Transparent Aqueous-Alcoholic Roll-On

| Oxyethylenated (20 EO) and oxypropylenated (5 PO) | 2.00 | g |
|---|---|---|
| cetyl alcohol | | |
| Hydroxyethylcellulose | 0.75 | g |
| Extract with Lipid A of Vitreoscilla filiformis (Example 2) | 0.0250 | g |
| Ethyl alcohol 95 vol. % denatured | 45.00 | g |
| Fragrance | qs | |
| Demineralized water | qs 100 | g |

### Example 9

### Anti-Hair Loss Gel Mask for Maintaining the Scalp

| | (% by |
|---|---|
| | weight) |
| Lactobacillus johnsonii (CNCM I-1225) | 0.05 |
| Hydroxypropylcellulose (Klucel H marketed by Hercules) | 1.00 |
| extract with Lipid A of Vitreoscilla filiformis (example 2) | 5.00 |
| Magnesium gluconate | 3.00 |
| Calcium linoleate | 2.00 |
| Blackcurrant seed oil | 5.00 |
| Evening primrose oil | 2.00 |
| Borage oil | 5.00 |
| Antioxidant | 0.05 |
| Vitamin C | 2.50 |
| Antioxidant | 0.05 |
| Isopropanol | 40.00 |
| Preservative | 0.30 |
| Water | qs 100.00 |

### Example 10

### Foot Cream

| Glyceryl mono/distearate/polyethylene glycol stearate | 8.00 | g |
|---|---|---|
| (100 EO) mixture | | |
| Cetyl alcohol | 2.50 | g |
| Microcrystalline wax/beeswax/polyethylene mixture | 1.00 | g |
| Liquid jojoba wax | 6.00 | g |
| Oxyethylenated myristyl myristate (3 EO) | 2.00 | g |
| extract with Lipid A of Vitreoscilla filiformis (example 2) | 0.4 | g |
| Fragrance, preservatives | Qs | |
| Demineralized water | qs 100 | g |

### Example 11

### Anti-Dandruff Lotion for the Scalp

| Ethyl alcohol 95 vol. % denatured | 55.00 | g |
|---|---|---|
| LPS extract with Lipid A of Vitreoscilla filiformis | 0.10 | g |
| (example 2) | | |
| Propylene glycol | 10.00 | g |
| Fragrance, dye | Qs | |
| Demineralized water | qs 100 | g |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for stimulating the defense systems of the skin against microorganisms, comprising administering to a subject in need of such treatment, a thus effective amount of Lipid A of Vitreoscilla filiformis having the following structure: (a) the compounds of formula (I): 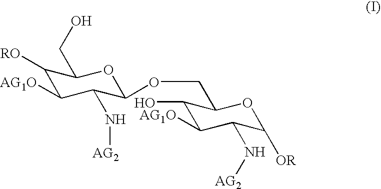
in which:
AG₁ is a 3-hydroxydecanoyl group,
AG₂ is a 3-dodecanoyloxydecanoyl group,
wherein R is a hydrogen atom or a group PO(OR′)₂, in which R′ is a hydrogen atom, a linear or branched, saturated or unsaturated C₁-C₆ alkyl radical, or a phenyl or benzyl group, and
(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that when more than one groups R, respectively R′, are present, they are identical to or different from one another.

2. The regime or regimen as defined by claim 1, comprising inducing the expression of anti-microbial peptides by the skin, the mucous membranes, the semi-mucous membranes and the scalp.

3. The regime or regimen as defined by claim 1, comprising preventing and/or reducing and/or inhibiting the proliferation of an unwanted microbial flora of the skin.

4. The regime or regimen as defined by claim 3, comprising the treatment of skin disorders linked to the development of a microorganism selected from the group consisting of Staphylococcus aureus, Streptococcus pyogenes, Pseudomonas aeruginosa, Escherichia coli, Clostridium pefringens, Clostridium difficile, Gardnerella vaginalis, Propionibacterium acnes, Klebsiella species, Streptopyogenes, Candida albicans, Malassezia furfur, Trichophyton mentagrophytes, Trichophyton interdigitale, Trichophyton rubrum, Trichophyton yaoundei, Tinea capitis, Tinea corporis, fungi and Aspergillus sp.

5. The regime or regimen as defined by claim 1, comprising the treatment of infectious complications of dermatological disorders; acne; infectious complications during cicatrization; dermatophytoses; candidiases; vaginoses; onychomycoses; scalp ringworm, body ringworm; skin disorders linked to therapies with antibiotics or anti-mycotic agents or initiated by hormone disturbances; dermatitis; erysipelas; or seborrheic dermatitis.

6. The regime or regimen as defined by claim 5, comprising preventing and/or treating microbial infections or superinfections of the skin.

7. The regime or regimen as defined by claim 5, comprising preventing and/or treating the infection or the superinfection of skin wounds or lesions.

8. The regime or regimen as defined by claim 5, comprising treating superinfected atopic dermatitis, impetiginous eczema, superinfected inflammatory acne or superinfected herpes.

9. The regime or regimen as defined by claim 8, comprising the treatment of superinfections during cicatrization and which are selected from ulcers, wounds and burns.

10. The regime or regimen as defined by claim 4, comprising the treatment of dermatophytoses selected from among scalp ringworm, body ringworm, athlete's foot, Hebra's eczema marginatum and herpes circinatus.

11. The regime or regimen as defined by claim 4, comprising the treatment of candidiases selected from among mucosal candidiases, vaginal candidiases, interdigital candidiases, candidiases linked to professions at risk or to diabetes.

12. The regime or regimen as defined by claim 4, comprising the treatment of dermatites selected from impetigo and superficial folliculitis.

13. The regime or regimen as defined by claim 1, comprising maintaining and/or restoring a normal skin ecoflora.

14. The regime or regimen as defined by claim 1, comprising preventing and/or treating dandruff conditions of the scalp.

15. The regime or regimen as defined by claim 1, comprising preventing and/or treating seborrheic conditions of the skin and of the scalp.

16. The regime or regimen as defined by claim 1, comprising maintaining oral hygiene.

17. The regime or regimen as defined by claim 1, comprising preventing and/or limiting unpleasant body odors.

18. The regime or regimen as defined by claim 1, wherein said extract is applied topically to the skin with or without body hair, the scalp, the mucous membranes and/or the semi-mucous membranes.

19. The regime or regimen as defined by claim 1, comprising treating a mammal.

20. The regime or regimen as defined by claim 19, comprising a veterinary treatment.

21. The regime or regimen as defined by claim 20, comprising treatment and/or prevention of disorders linked to staphylococcal, streptococcal or mycotic infections.

22. The regime or regimen as defined by claim 1, wherein said fraction is administered in combination with a probiotic and/or a prebiotic.

23. The regime or regimen as defined by claim 22, wherein said fraction is administered in combination with a probiotic and a prebiotic.

24. The regime or regimen as defined by claim 22, wherein said probiotic is selected from among Saccharomyces, Yarrowia, Kluyveromyces, Torulaspora, Schizosaccharomyces pombe, Debaromyces, Candida, Pichia, Aspergillus and Penicillium, and bacteria of the genus Bifidobacterium, Bacteroides, Fusobacterium, Melissococcus, Propionibacterium, Enterococcus, Lactococcus, Staphylococcus, Peptostrepococcus, Bacillus, Pediococcus, Micrococcus, Leuconostoc, Weissella, Aerococcus, Oenococcus or Lactobacillus.

25. The regime or regimen as defined by claim 24, wherein said probiotic is selected from among Lactobacillus johnsonii, Lactobacillus reuteri, Lactobacillus rhamnosus, Lactobacillus paracasei, Lactobacillus casei or Bifidobacterium bifidum, Bifidobacterium breve, Bifidobacterium longum, Bifidobacterium animalis, Bifidobacterium lactis, Bifidobacterium infantis, Bifidobacterium adolescentis or Bifidobacterium pseudocatenulatum, and mixtures thereof.

26. The regime or regimen as defined by claim 24, wherein said probiotic is present at a concentration ranging from 10³ to 10¹² cfu of microorganisms per gram of a support therefor.

27. The regime or regimen as defined by claim 23, wherein said prebiotic is selected from fructooligosaccharides, inulin and isomaltooligosaccharides.

28. The regime or regimen as defined by claim 1, comprising administering Lipid A of Vitreoscilla filiformis having the following structure:
(a) the compounds of formula (I): 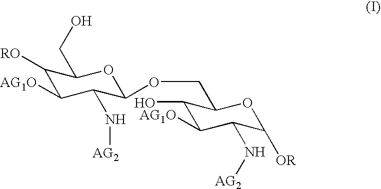
in which:
AG₁ is a 3-hydroxydecanoyl group,
AG₂ is a 3-dodecanoyloxydecanoyl group,
wherein R is a hydrogen atom or a group PO(OR′)₂, in which R′ is a hydrogen atom, a linear or branched, saturated or unsaturated C₁-C₆ alkyl radical, or a phenyl or benzyl group, and
(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that when one or more groups R, respectively R′, are present, they are identical to or different from one another,
before, simultaneously with or after the administration of a peeling agent.

29. The regime or regimen as defined by claim 28, wherein said peeling agent is selected from saturated and unsaturated monocarboxylic acids, saturated and unsaturated dicarboxylic acids, saturated and unsaturated tricarboxylic acids; alpha-hydroxy acids and beta-hydroxy acids of monocarboxylic acids; alpha-hydroxy acids and beta-hydroxy acids of dicarboxylic acids; alpha-hydroxy acids and beta-hydroxy acids of tricarboxylic acids; keto acids, alpha-keto acids, beta-keto acids of polycarboxylic acids, of polyhydroxymonocarboxylic acids, of polyhydroxydicarboxylic acids, of polyhydroxytricarboxylic acids; and (3-hydroxy-2-pentylcyclopentyl)acetic acid, pyruvic acid, gluconic acid, glucuronic acid, oxalic acid, malonic acid, succinic acid, acetic acid, gentisic acid, cinnamic acid, azelaic acid; phenol, resorcinol; urea and derivatives thereof; oligofucoses; jasmonic acid and derivatives thereof; trichloroacetic acid; retinoids such as retinol or retinoic acid; adapalene; extract of Saphora japonica; resveratrol; and also salts and derivatives thereof, the cis or trans forms thereof, racemic mixtures, and the dextrorotatory or levorotatory forms of the abovementioned agents; glycosidases, stratum corneum chymotryptic enzyme (SCCE) or other proteases (trypsin, chymotrypsin-like); mineral salt chelating agents, EDTA; N-acyl-N,N′,N′-ethylenediaminetriacetic acid; aminosulfonic compounds, (N-2-hydroxyethylpiperazine-N-2-ethane)sulfonic acid (HEPES); derivatives of 2-oxothiazolidine-4-carboxylic acid (procysteine); derivatives of alpha-amino acids of glycine type, sodium methyl glycine diacetate; honey; sugar derivatives, O-octanoyl-6-D-maltose, O-linoleyl-6-D-glucose and N-acetylglucosamine; extracts of laminaria, Laminaria saccharina and Laminaria ochrolenca, glyceryl trilactate, siliceous salicylate derivatives, 5-acylsalicylic acid salts, active agents with effects on transglutaminase, and an extract of Ficus opuntia indica blossom.

30. A cosmetic skin-peeling process comprising the application, simultaneously with or following the application of peeling agent, of Lipid A of Vitreoscilla filiformis having the following structure: (a) the compounds of formula (I): 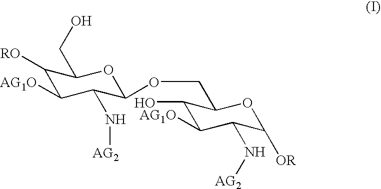
in which:
AG₁ is a 3-hydroxydecanoyl group,
AG₂ is a 3-dodecanoyloxydecanoyl group,
wherein R is a hydrogen atom or a group PO(OR′)₂, in which R′ is a hydrogen atom, a linear or branched, saturated or unsaturated C₁-C₆ alkyl radical, or a phenyl or benzyl group, and
(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that when more than one groups R, respectively R′, are present, they are identical to or different from one another.

31. A regime or regimen for preventing microbial proliferation in cell or organotypic cultures, comprising administering thereto Lipid A of Vitreoscilla filiformis having the following structure: (a) the compounds of formula (I): 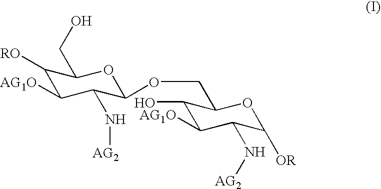
in which:
AG₁ is a 3-hydroxydecanoyl group,
AG₂ is a 3-dodecanoyloxydecanoyl group,
wherein R is a hydrogen atom or a group PO(OR′)₂, in which R′ is a hydrogen atom, a linear or branched, saturated or unsaturated C₁-C₆ alkyl radical, or a phenyl or benzyl group, and
(b) in the event that R is a group PO(OH)₂ in formula (I) above, an inorganic salt of the compound of formula (I), or a primary, secondary or tertiary amine salt of the compound of formula (I), or a phosphoethanolamine salt of the compound of the formula (I), with the proviso that when more than one groups R, respectively R′, are present, they are identical to or different from one another.

32. The regime or regimen as defined by claim 31, comprising the septic preparation of epidermal and/or skin models.

33. The regime or regimen as defined by claim 31, comprising the septic preparation of skin explants or of hair grafts.

34. A skin-peeling composition comprising the Lipid A as defined in claim 1 and a skin-peeling agent.

35. A composition comprising the Lipid A as defined in claim 1 and a probiotic and/or a prebiotic.
